(19) United States
(12) Patent Application Publication
(10) Pub. No.: US 2019/0321548 A1
(43) Pub. Date: Oct. 24, 2019

(54) CATHETER HAVING A CANNULA WITH BOTH AN END OPENING AND A PLURALITY OF PERFORATIONS IN THE CANNULA WALL

(71) Applicant: Nathan Montgomery Cowan, Lehi, UT (US)

(72) Inventor: Nathan Montgomery Cowan, Lehi, UT (US)

(21) Appl. No.: 16/388,803

(22) Filed: Apr. 18, 2019

Related U.S. Application Data
(60) Provisional application No. 62/659,302, filed on Apr. 18, 2018.

(51) Int. Cl. A61M 5/158; A61M 39/02; A61M 5/172; A61M 5/142

(52) U.S. Cl. CPC A61M 5/158; A61M 39/0208; A61M 25/02; A61M 5/14248; A61M 5/1723

(57) ABSTRACT

An improved catheter for the dispensing of pharmaceutical fluids, such as insulin, has a cannula with not only a conventional end opening, but also a plurality of micro perforations in the cannula wall, which prevent flow blockages of pharmaceutical fluids if the cannula becomes kinked near its open end. The perforations are located in a portion of the cannula wall that is intended to be installed beneath the patient's skin. Thus, if the end opening becomes blocked because of a kink in the cannula near the open end, the pharmaceutical fluid can still enter the patients blood stream through the micro perforations in the cannula wall. Perforations are preferably formed in a spiral pattern along a portion of the cannula to prevent diametrically opposed perforations that might compromise cannula wall strength.

### BACKGROUND OF THE INVENTION

### 1. Field of the Invention

The present invention relates, generally, to catheters and, more particularly, to the flexible cannulae of catheters used to deliver a fluid, such as insulin, into the body and to extract blood from the body.

### 2. History of the Prior Art

A cannula (from Latin “little reed”) is a tube that can be inserted into the body, often for the delivery or removal of fluid or for the gathering of data. In simple terms, a cannula can surround the inner or outer surfaces of a trocar needle thus extending the effective needle length by at least half the length of the original needle. It is also called an intravenous, or IV, cannula. The size of cannulae typically ranges from 14 to 24 gauge. Different-sized cannula are coded with different colors.

A cannula normally comes with a trocar attached. A trocar is a sharp-pointed surgical instrument fitted with a cannula that is used to puncture the body in order to insert the cannula into that location. Insulin pumps are becoming more popular as the technology improves and additional features are added. Some pumps are now available that work in conjunction with continuous glucose monitors that can alert the user to high or low blood glucose levels if programmed to do so.

Type 1 diabetes, once known as juvenile diabetes or insulin-dependent diabetes, is a chronic condition in which the pancreas produces little or no insulin, the hormone that is required to enable glucose to enter cells to produce energy. Different factors, including genetics and some viruses, may contribute to type 1 diabetes. Although type 1 diabetes usually appears during childhood or adolescence, it can also develop in adults. Despite active research, type 1 diabetes has no cure. Treatment focuses on managing blood sugar levels with insulin, diet and lifestyle to prevent complications. Insulin pumps are commonly used to deliver a controlled flow of insulin to the bloodstream of people with Type 1 diabetes. Insulin pumps are also becoming a treatment of choice for many with Type 2 diabetes, as well. Most insulin pumps are small devices about the size of a cell phone. An exception is the OmniPod®, which is even smaller and is attached directly to the skin, with no tubing necessary. A conventional insulin pump is typically clipped onto the waistband of individual's pants or skirt, or is placed in a pocket. A length of plastic tubing connects the insulin reservoir within the pump to a type of catheter known as an infusion set. FIG. 1 shows a typical infusion set 100. This particular infusion set is a Tandem Diabetes t:90 Infusion Set having a cannula 101 that is 9 mm in length. An insertion needle 102 extends through the cannula 101 and passes through the open end of the cannula 101 so that the sharp end 103 of the needle 102 is exposed. The opposite blunt end 104 of the needle 102 is embedded in a hilt 105, that facilitates removal of the needle 102 from the cannula 101 once the sharp end 103 of the needle 102 and the cannula have been inserted beneath the patient's epidermis. The non-insertion end 106 of the cannula 101 is embedded within an injection molded body 107 having a socket 108 into which both the hilt 104 and a tubing connector (not shown) fit. Flexible wings 109A and 109B on the body 107 lock the tubing connector into place and also facilitate release of the tubing connector when squeezed together by the patient. The body 107 is bonded to the upper surface 110 of a generally circular flexible sheet 111. The flexible sheet 111 has adhesive on its lower surface which enables it to be adhered to the patient's skin at the infusion site. Referring now to FIG. 2, the infusion set 100 is shipped with a protective sheath 201 covering the sharp end 103 of the insertion needle 102. The infusion set works the same way as an intravenous line does. A needle is housed inside the cannula, which is a tiny plastic tube, the end of which is usually placed under the skin in the subcutaneous fat. The needle is used to puncture the skin so that the cannula can be inserted. After insertion, the needle is removed and the cannula remains in place. The cannula is commonly inserted into the wrist, abdomen or buttocks and needs to be changed every 48 hours. Although there are many types of infusion sets, they can be categorized into two main groups—those which are “angled” and those which are “straight”. Angled sets are generally inserted into the body at a 30-degree angle. They sit laterally under the skin. The cannula and needle are available in different lengths. Straight sets, on the other hand, are inserted perpendicularly at a 90-degree angle. The cannula is available in different lengths. Irrespective of the type of infusion set used, the user programs the insulin pump to deliver insulin at a slow, continuous (basal) rate as well as in supplemental (bolus) doses before meals and to correct for high blood glucose. Basal and bolus dosing most closely resembles how the pancreas releases insulin in a person without diabetes.

Cannulae are also used in peripheral venous catheters (PVCs). Using such a catheter, a flexible cannula is placed into a peripheral vein for intravenous therapy using medication fluids. In addition, cannula can also be used to draw blood. A peripheral venous catheter is the most commonly used vascular access in medicine. It is given to most emergency department and surgical patients, and before some radiological imaging techniques using radiocontrast, for example. In the United States, alone, more than 30 million patients receive a peripheral venous line each year.

As with an infusion set, the cannula is introduced into the vein as a sheath covering a trocar, which is subsequently removed. The cannula remains in place. The catheter is then affixed behind the insertion site by taping it to the patient's skin or by using an adhesive dressing.

A peripheral venous catheter is usually placed in a vein on the hand or arm. It should be distinguished from a central venous catheter which is inserted in a central vein (usually in the internal jugular vein of the neck or the subclavian vein of the chest), or an arterial catheter which can be placed in a peripheral or central artery. In children, a topical anaesthetic gel (such as lidocaine) may be applied to the insertion site to facilitate placement.

One of the common problems associated with cannulae used to dispense insulin into body tissues of diabetics and medications into a vein is that a cannula can, like a garden hose, become kinked. With normal use, cannulae typically become kinked more than half the time. For some users, as many as eighty percent of cannulae may develop a flow-limiting kink during a typical forty-eight hour use period. Typically, the kinking occurs within several millimeters from the open end. When that occurs, fluid flow can become severely reduced or completely blocked. When the flow of insulin is severely reduced or blocked, severely elevated glucose levels can rapidly occur, causing a medical emergency involving diabetic ketoacidosis (DKA) or hyperglycemic hyperosmolar nonketotic syndrome (HHNS), also referred to as hyperglycemic hyperosmolar state). These two acute complications of diabetes can result in increased morbidity and mortality if not efficiently and effectively treated. Mortality rates are 2-5% for DKA and 15% for HHS, and mortality is usually a consequence of the underlying precipitating cause(s) rather than a result of the metabolic changes of hyperglycemia.

### SUMMARY OF THE INVENTION

The present invention provides an improved catheter for the dispensing of critical pharmaceutical fluids, such as insulin, as well as other medications. The improved catheter is equipped with a cannula that has, in addition to a conventional end opening, a plurality of micro perforations in the cannula wall, which prevent flow blockages of pharmaceutical fluids if the cannula becomes kinked. The perforations are located in a portion of the cannula wall that is intended to be installed beneath the patient's skin. Thus, if the end opening becomes blocked because of a kink in the cannula near the open end, the pharmaceutical fluid can still enter the patients blood stream through the micro perforations in the cannula wall. In essence, the improved cannula is much like a garden soaker hose with the end plug removed, but on a much smaller scale. In addition, whereas the garden soaker hose is perforated along its entire length, the improved cannula is perforated only near the open end where it is certain to be embedded beneath the patient's epidermis. If the cannula were perforated along its entire length, much of the pharmaceutical fluid would escape before having an opportunity to enter the patient's blood stream, thereby preventing proper quantities of the fluids to enter the patient's bloodstream. The micro perforations in the cannula wall are preferably formed using laser ablation. Although they can also be formed using micro mechanical punching, there is some danger that stray micro chads or punchings could enter the patient's bloodstream and cause clotting. Micro perforations formed using laser ablation are unlikely to leave any potentially dangerous particles within the cannula that could enter the blood stream when the pharmaceutical fluid begins to flow.

### BRIEF DESCRIPTION OF THE DRAWINGS

FIG. 1 is a top plan view of a conventional infusion set;

FIG. 2 is a top plan view of a conventional infusion set from which the protective sheath covering the needle has been removed;

FIG. 3 is a top plan view of a first embodiment improved infusion set with the protective sheath covering the needle removed in order to show a first spiral pattern of perforations along a forward portion of the cannula;

FIG. 4 is an isometric view of a forward portion of the cannula of the first embodiment improved infusion set;

FIG. 5 is a front elevational view of the cannula of FIG. 4, showing the forward opening and the pattern of apertures that are hidden in this view;

FIG. 6 is a top plan view of a second embodiment improved infusion set with the protective sheath covering the needle removed in order to show a second spiral pattern of perforations along a forward portion of the cannula;

FIG. 7 is an isometric view of a forward portion of the cannula of the second embodiment improved infusion set;

FIG. 8 is a front elevational view of the cannula of FIG. 7, showing the forward opening and the pattern of apertures that are hidden in this view as opening of the cannula;

FIG. 9 is a top plan view of a conventional peripheral venous catheter;

FIG. 10 is a top plan view of the peripheral venous catheter of FIG. 9, which has been fitted with the improved cannula; and

FIG. 11 is a closeup view of the encircled needle portion 10 of FIG. 9.

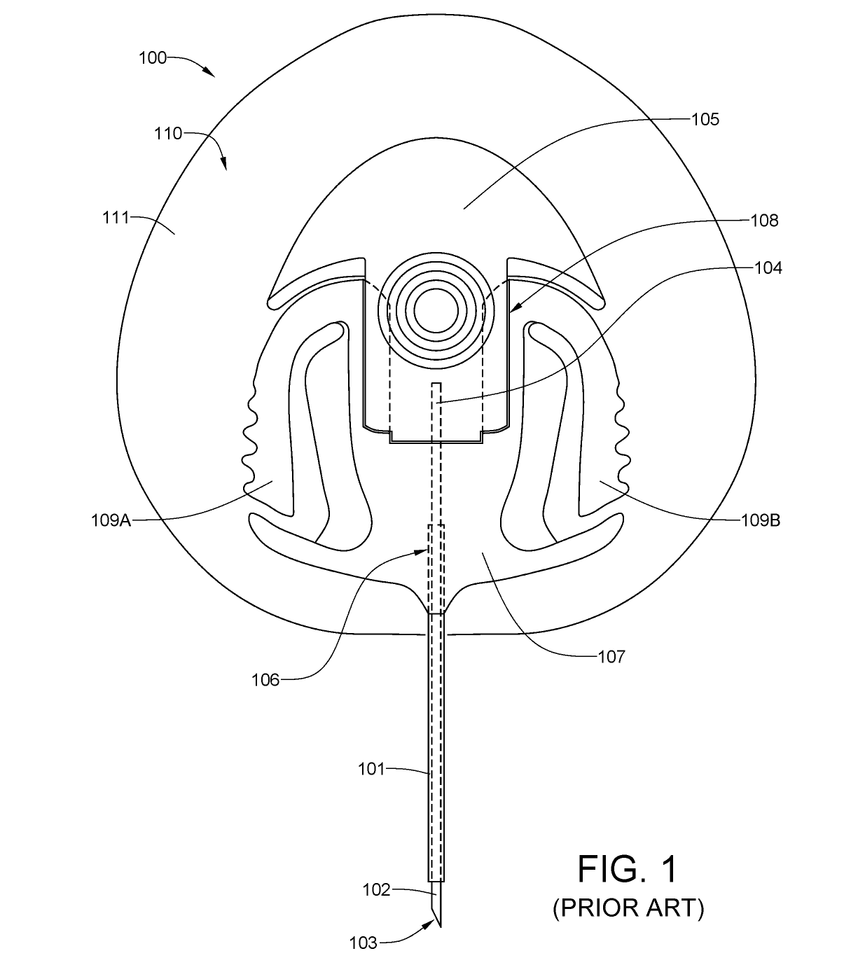

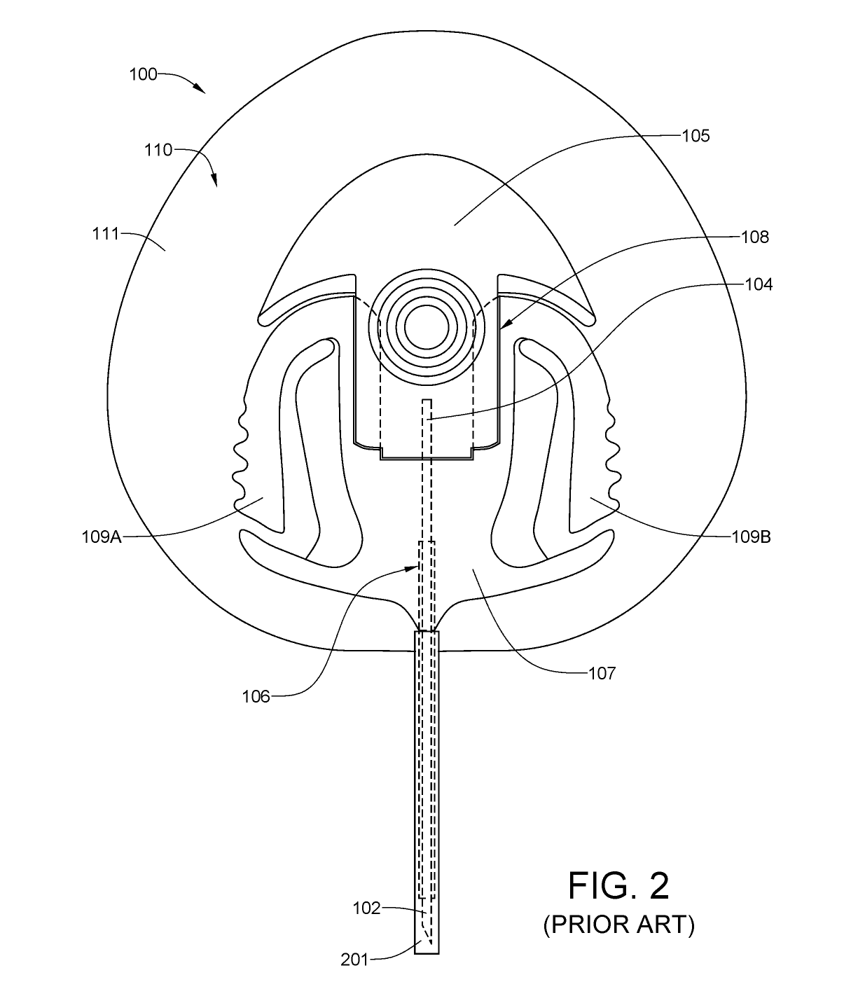

### DETAILED DESCRIPTION OF THE PREFERRED EMBODIMENT

The improved catheter will now be described with reference to the attached drawing figures. It should be understood that, although the drawings are not necessarily drawn to exact scale, they are intended to fully convey an understanding of the invention with respect to how it is made and how it is used.

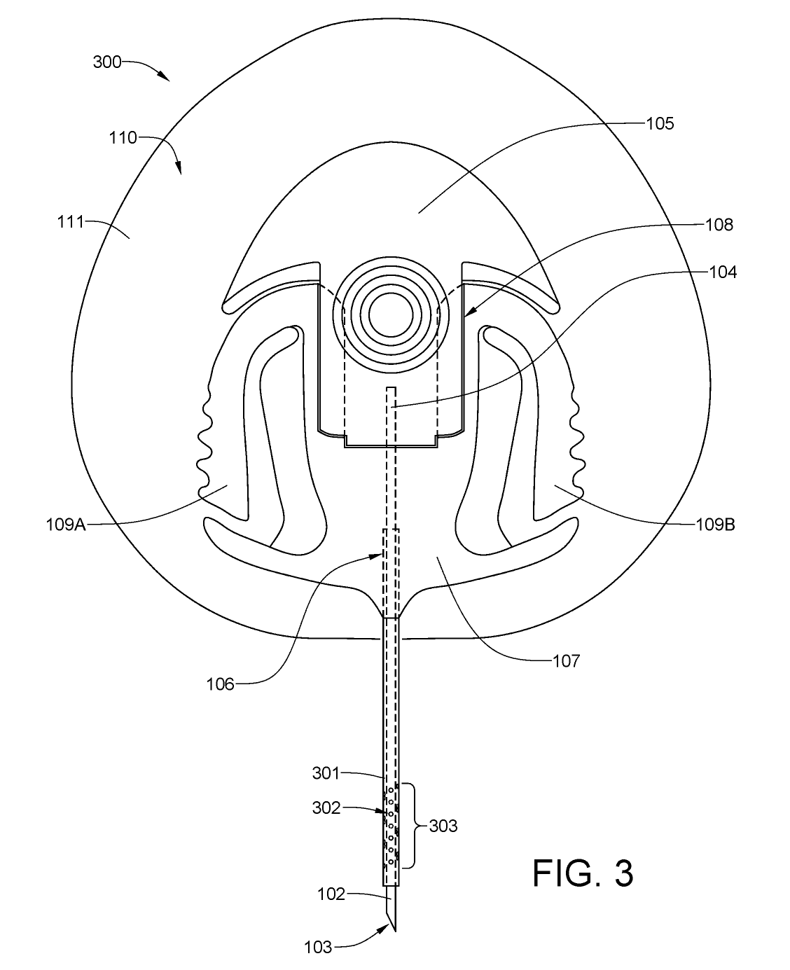

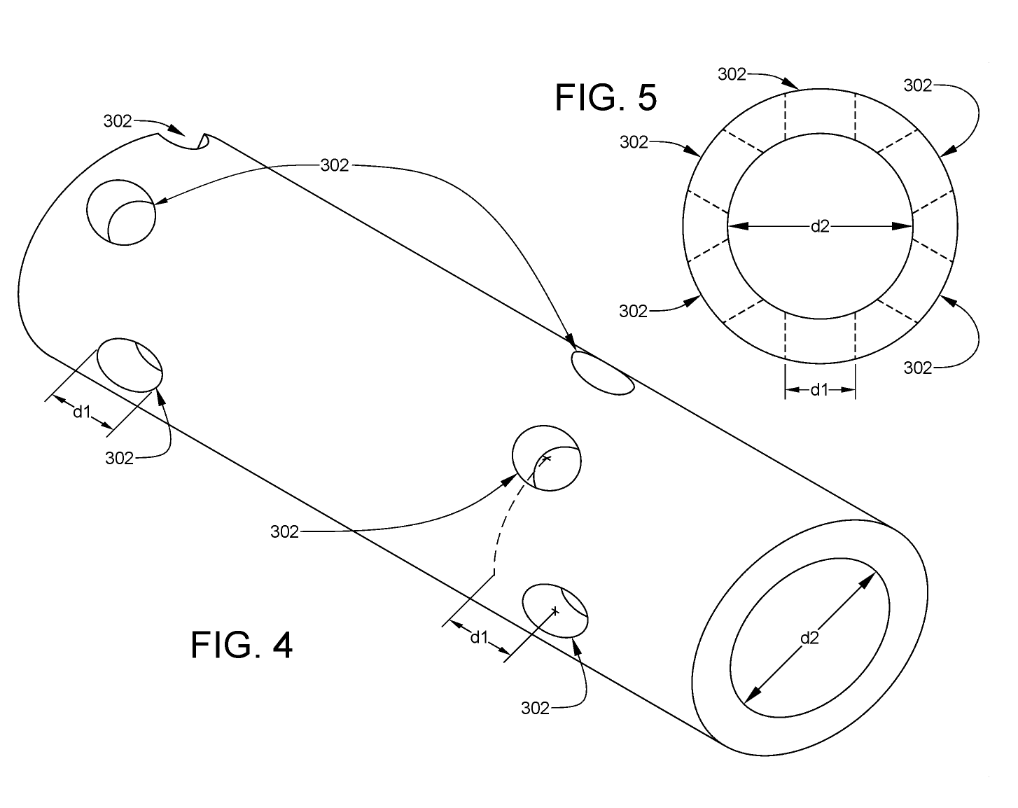

Referring now to FIGS. 3, 4 and 5, a first embodiment improved infusion set 300 has a cannula 301, the forward end of which is perforated with a plurality of perforations 302 in a first spiral pattern 303. Each succeeding perforation 302 in the pattern is offset rearward by a distance that is about equal to the diameter d1 of each perforation. The diameter d1 of each perforation is about one-fourth the diameter d2 of the forward opening 401 of the cannula 301. The perforations 302 are preferably formed by laser ablation of the cannula material. The perforations can be made with the needle is inserted within the cannula. This will prevent the laser beam from perforating both sides of the cannula in a diametrically opposed arrangement. Diametrically-opposed perforations are avoided to limit the reduction in wall strength at any given position along the cannula's length. There is a gap of at least 5 mm between the rearward termination of the first spiral pattern 303 and the portion of the cannula 301 that remains exposed when the forward end is inserted into the patient's body. The gap prevents leakage of insulin outside the patient's body.

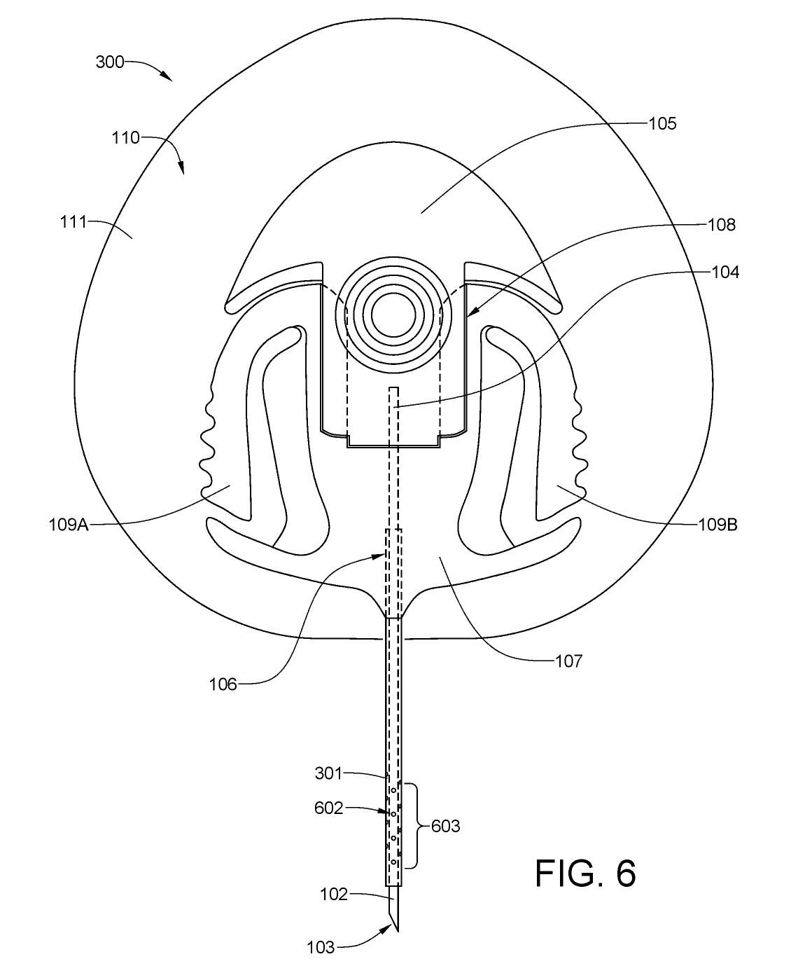

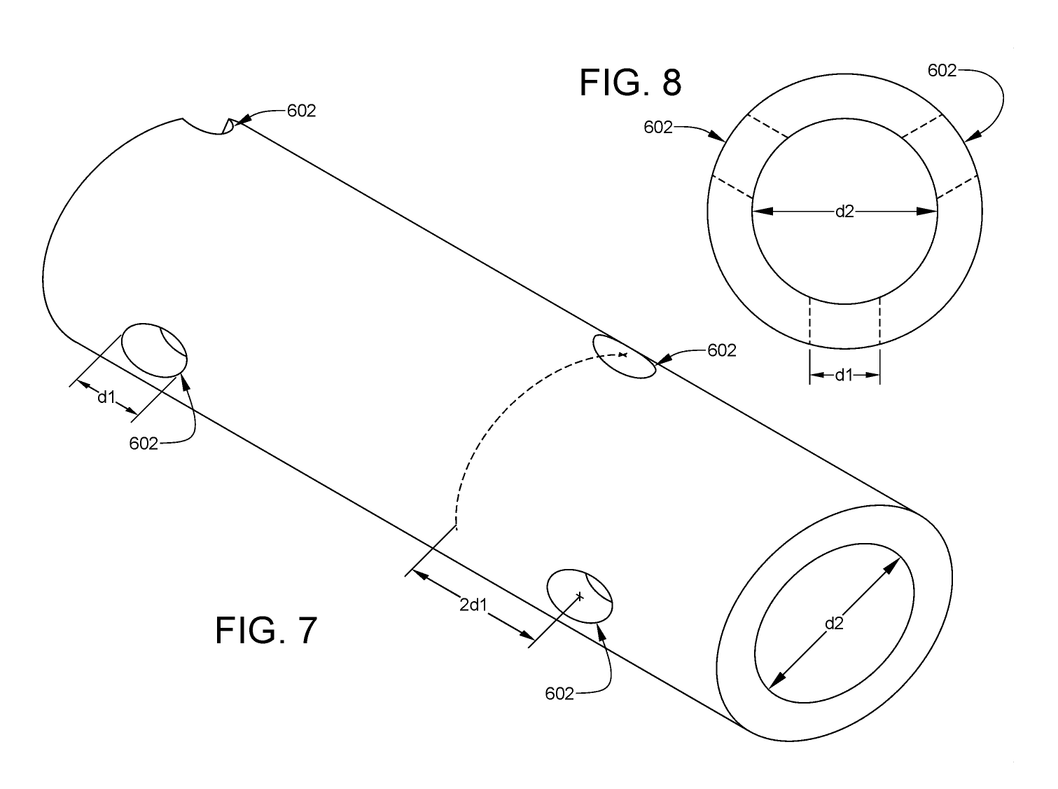

Referring now to FIGS. 6, 7 and 8, a second embodiment improved infusion set 600 has a cannula 601, the forward end of which is perforated with a plurality of perforations 602 in a second spiral pattern 603. Each succeeding perforation 602 in the pattern is offset rearward by a distance that is about equal to twice the diameter d1 of each perforation. The diameter d1 of each perforation is about one-fourth the diameter d2 of the forward opening 401 of the cannula 601. The perforations 602 are preferably formed by laser ablation of the cannula material. The perforations can be made with the needle is inserted within the cannula. This will prevent the laser beam from perforating both sides of the cannula in a diametrically opposed arrangement. Diametrically-opposed perforations are avoided to limit the reduction in wall strength at any given position along the cannula's length. There is a gap of at least 5 mm between the rearward termination of the second spiral pattern 603 and the portion of the cannula 601 that remains exposed when the forward end is inserted into the patient's body. The gap prevents leakage of insulin outside the patient's body.

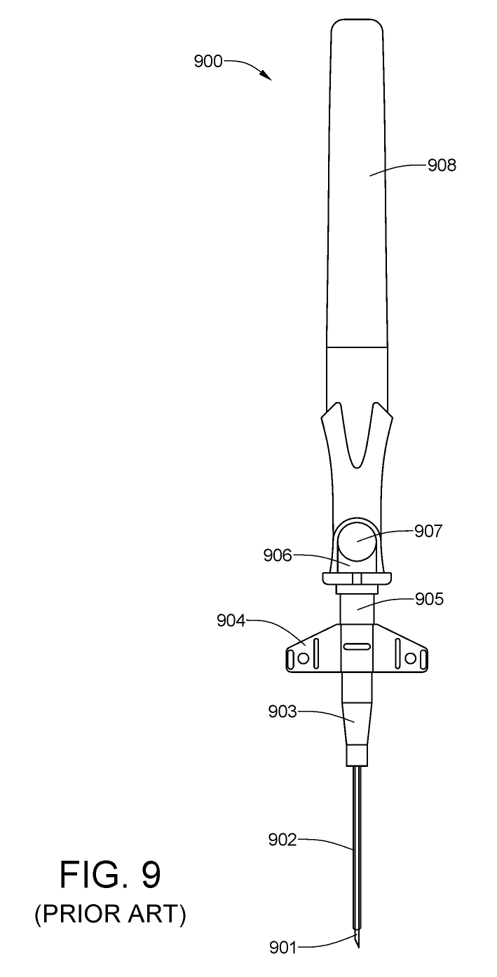

Referring now to FIG. 9, a conventional peripheral venous catheter 900 has a sharpened needle 901, a flexible cannula 902, a fore hub portion 903, wings 904, an aft hub portion 905, a flashback chamber 906, a needle grip 907, and a needle safety cover 908.

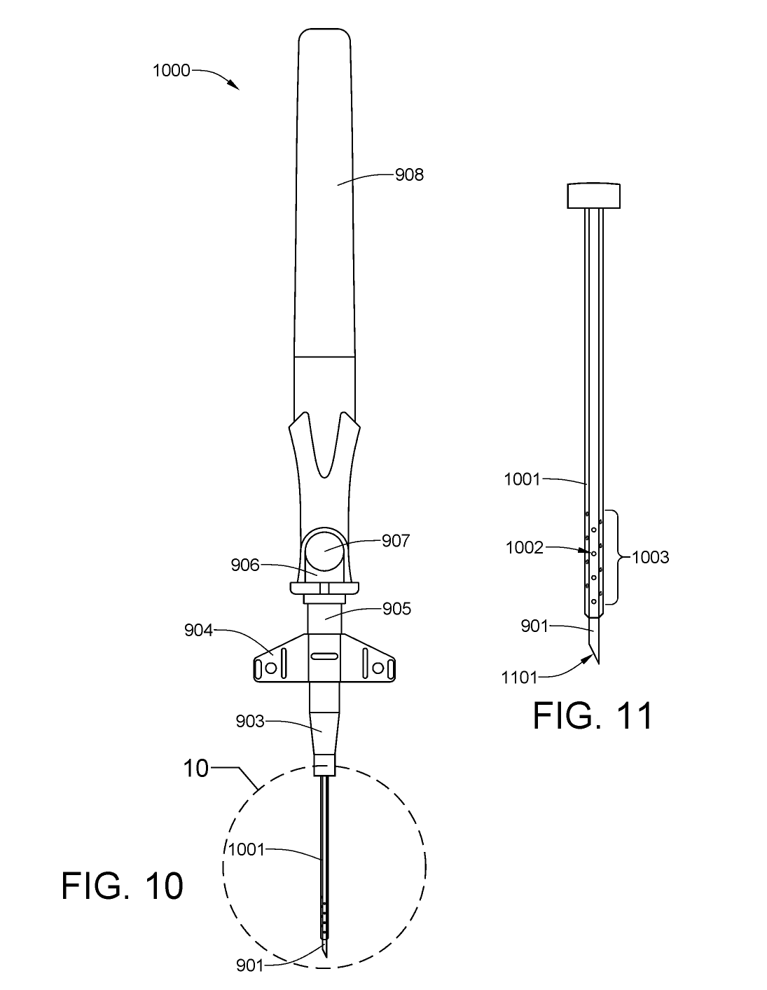

Referring now to FIGS. 10 and 11, the catheter of FIG. 9 has been modified to include a cannula 1001 having a forward end of which is perforated with a plurality of perforations 1002 in a first spiral pattern 1003. As with the cannula 301 for the infusion set 300, each succeeding perforation 1002 in the pattern is offset rearward by a distance that is about equal to the diameter of each perforation. The diameter of each perforation is about one-fourth the diameter of the forward opening of the cannula 1001. The perforations 1002 are preferably formed by laser ablation of the cannula material. The perforations can be made with the needle 901 is inserted within the cannula 1001. This will prevent the laser beam from perforating both sides of the cannula in a diametrically opposed arrangement. Diametrically-opposed perforations are avoided to limit the reduction in wall strength at any given position along the cannula's length. There is a gap of at least 5 mm between the rearward termination of the first spiral pattern 303 and the portion of the cannula 1001 that remains exposed when the forward end is inserted into the patient's body. The gap prevents leakage of fluids and blood outside the patient's body.

Although only several embodiments of the improved catheter have been shown and described, it will be obvious to those having ordinary skill in the art that changes and modifications may be made thereto without departing from the scope and the spirit of the invention as hereinafter claimed.

What is claimed is:

1. An improved catheter for dispensing pharmaceutical fluids into a patient's body, said catheter of a type having a thermoplastic body in which is embedded a first end of a cannula, said cannula also having an open free second end, a removable needle disposed within the cannula and extending beyond the open free end for insertion of the cannula's second end into a patient's body, said thermoplastic body also having a socket in communication with said cannula, said socket being couplable to a pharmaceutical fluid line, wherein the improvement comprises a plurality of perforations within a forward portion of said cannula, said forward portion intended for complete insertion within the patient's body; wherein said plurality of perforations provide an outlet for infused pharmaceutical fluids, even if the cannula becomes kinked near its open free second end.

2. The improved catheter of claim 1, wherein said plurality of perforations are arranged in a spiral pattern about the cannula.

3. The improved catheter of claim 2, where a central axis of each perforation is perpendicular to a central axis of the cannula.

4. The improved catheter of claim 3, wherein the axes of no two perforations intersect the central axis of the cannula at the same point.

5. The improved catheter of claim 4, wherein said perforations are cylindrical and of a same size, and wherein adjacent perforations are offset along the central axis of the cannula by at least a distance equal to a diameter of the perforations.

6. The improved catheter of claim 4, wherein said perforations are cylindrical and of a same size, and wherein adjacent perforations are offset along the central axis of the cannula by at least a distance equal to twice a diameter of the perforations.

7. The improved catheter of claim 1, wherein there is a gap of at least 5mm between a rearward termination of the spiral pattern and a portion of the cannula that will remain exposed when the forward end of the cannula is inserted into the patient's body, said gap preventing leakage of the pharmaceutical fluid outside the patient's body.

8. An improved cannula for use with infusion sets and peripheral venous catheters, wherein the improvement comprises a plurality of perforations within a forward portion of said cannula, said forward portion intended for complete insertion within a patient's body; and wherein said plurality of perforations provide an outlet for infused pharmaceutical fluids, even if the cannula becomes kinked near its open free end that has been inserted with the patient's body.

9. The improved cannula of claim 8, wherein said plurality of perforations are arranged in a spiral pattern about the cannula.

10. The improved cannula of claim 9, where a central axis of each perforation is perpendicular to a central axis of the cannula.

11. The improved cannula of claim 10, wherein the axes of no two perforations intersect the central axis of the cannula at the same point.

12. The improved cannula of claim 10, wherein said perforations are cylindrical and of a same size, and wherein adjacent perforations are offset along the central axis of the cannula by at least a distance equal to a diameter of the perforations.

13. The improved cannula of claim 10, wherein said perforations are cylindrical and of a same size, and wherein adjacent perforations are offset along the central axis of the cannula by at least a distance equal to twice a diameter of the perforations.

14. The improved cannula of claim 8, wherein there is a gap of at least 5mm between a rearward termination of the spiral pattern and a portion of the cannula that will remain exposed when the forward end of the cannula is inserted into the patient's body, said gap preventing leakage of the pharmaceutical fluid outside the patient's body.

15. A catheter for dispensing pharmaceutical fluids into a patient's body, said catheter comprising:
a thermoplastic body having a socket couplable to a pharmaceutical fluid line;
a cannula having a first end embedded in the thermoplastic body and in communication with said socket, said cannula also having an open free second end that can be inserted into the patient's body, said cannula having a plurality of perforations within a forward portion thereof, said forward portion intended for complete insertion within the patient's body; wherein said plurality of perforations provide an outlet for infused pharmaceutical fluids, even if the cannula becomes kinked near its open free second end; and
a removable needle disposed within the cannula and extending beyond the open free second end for insertion of the cannula's second end into a patient's body.

16. The improved catheter of claim 15, wherein said plurality of perforations are arranged in a spiral pattern about the cannula.

17. The improved catheter of claim 16, where a central axis of each perforation is perpendicular to a central axis of the cannula and the axes of no two perforations intersect the central axis of the cannula at the same point.

18. The improved catheter of claim 17, wherein said perforations are cylindrical and of a same size, and wherein adjacent perforations are offset along the central axis of the cannula by at least a distance equal to a diameter of the perforations.

19. The improved catheter of claim 17, wherein said perforations are cylindrical and of a same size, and wherein adjacent perforations are offset along the central axis of the cannula by at least a distance equal to twice a diameter of the perforations.

20. The improved catheter of claim 15, wherein there is a gap of at least 5mm between a rearward termination of the spiral pattern and a portion of the cannula that will remain exposed when the forward end of the cannula is inserted into the patient's body, said gap preventing leakage of the pharmaceutical fluid outside the patient's body.
